(19) United States
(12) Patent Application Publication
(10) Pub. No.: US 2014/0370551 A1
(43) Pub. Date: Dec. 18, 2014

(54) PRODUCTION OF SUGARS AND CO-PRODUCTS FROM CELLULOSIC WASTE STREAMS

(71) Applicant: API Intellectual Property Holdings, LLC, Atlanta, GA (US)

(72) Inventors: Theodora RETSINA, Atlanta, GA (US); Kimberly NELSON, Atlanta, GA (US)

(21) Appl. No.: 14/305,076

(22) Filed: Jun. 16, 2014

Related U.S. Application Data
(60) Provisional application No. 61/836,014, filed on Jun. 17, 2013.

(51) Int. Cl. C13K 1/02; C12P 19/14; C12P 19/02

(52) U.S. Cl. CPC C13K 1/02; C12P 19/02; C12P 19/14
USPC 435/99; 127/37; 568/672; 435/136; 435/165; 435/157; 435/160; 435/158; 435/150; 435/126; 435/139; 526/352; 526/351; 528/354; 528/308.1; 526/317.1; 528/85; 528/165; 528/332; 526/295; 526/344; 526/346; 526/341; 528/10

(57) ABSTRACT

This disclosure provides a business method and system for generating sugars and recycling a non-biomass component from a waste stream. In some embodiments, a waste stream comprising cellulose and a non-biomass component is saccharified to produce glucose, followed by recovery of the glucose and non-biomass component, which may be recycled to another site associated with production of a cellulose-containing product that contains the non-biomass component. In certain scenarios, the waste stream is generated at a first location, cellulose pretreatment (if desired) and hydrolysis are conducted at a second location, and the non-biomass component is recycled to the first location or a third location. The non-biomass component may include metals, metal oxides, salts, organic compounds, inorganic compounds, oligomers, or polymers, for example.

### PRIORITY DATA

This patent application is a non-provisional application with priority to U.S. Provisional Patent App. No. 61/836,014, filed Jun. 17, 2013, which is hereby incorporated by reference herein in its entirety.

### FIELD

The present invention generally relates to processes for converting cellulose-containing waste streams into fermentable sugars and co-products.

### BACKGROUND

Biomass refining (or biorefining) is becoming more prevalent today. Cellulose fibers and sugars, hemicellulose sugars, lignin, syngas, and derivatives of these intermediates are being used by many companies for chemical and fuel production. Indeed, we now are observing the commercialization of integrated biorefineries that are capable of processing incoming biomass much the same as petroleum refineries now process crude oil. Underutilized lignocellulosic biomass feedstocks have the potential to be much cheaper than petroleum, on a carbon basis, as well as much better from an environmental life-cycle standpoint.

Lignocellulosic biomass is the most abundant renewable material on the planet and has long been recognized as a potential feedstock for producing chemicals, fuels, and materials. Lignocellulosic biomass normally comprises primarily cellulose, hemicellulose, and lignin. Cellulose and hemicellulose are natural polymers of sugars, and lignin is an aromatic/aliphatic hydrocarbon polymer reinforcing the entire biomass network.

Some forms of biomass are rich in cellulose but contain little, if any, hemicellulose or lignin. Examples of such biomass include municipal solid waste streams, industrial waste streams, consumer waste, recycled materials, and the like. There is currently a need in the art for processes and apparatus that can convert these waste streams into fermentable sugars, such as glucose, or other valuable products (or materials to be reused). The sugars can be fermented to ethanol or other products.

### SUMMARY

The present invention addresses the aforementioned needs in the art.

In some variations, the invention provides a process for generating sugars from a waste stream, the process comprising:

(a) providing a waste stream comprising cellulose and a non-biomass component;

(b) introducing the waste stream to a saccharification reactor under effective conditions to saccharify at least some of the cellulose to produce glucose, wherein the non-biomass component is not substantially degraded in the saccharification reactor;

(c) recovering or further processing the glucose; and

(d) recovering at least some of the non-biomass component.

In some embodiments, the non-biomass component is selected from the group consisting of metals, metal oxides, salts, organic compounds, inorganic compounds, oligomers, polymers, and combinations thereof. In certain embodiments, the non-biomass component consists of or includes a polymer selected from the group consisting of polyethylene, polypropylene, polylactide, polyethylene glycol, polyethylene terephthalate, polyacrylic acid, polyurethanes, synthetic rubber, phenol formaldehyde resin, neoprene, nylon, polyvinyl chloride, polystyrene, polyacrylonitrile, silicone, and combinations thereof.

In some embodiments, the waste stream is derived from clothing manufacturing, in which case the cellulose may be in the form of cotton or other cellulose fibers. Various non-biomass components may be present, including dyes, inks, or natural or synthetic materials.

In some embodiments, step (b) utilizes cellulase enzymes. In these or other embodiments, step (b) utilizes an acid or base catalyst. Step (b) may be conducted at a solids concentration of from about 1 wt % to about 25 wt % on a dry basis, such as from about 2 wt % to about 10 wt % on a dry basis, for example.

The process may further comprise a refining step, prior to or during step (b), to reduce average particle size of the waste stream. In some embodiments, the refining step includes mechanical refining, chemical refining, thermomechanical refining, chemithermomechanical refining, or a combination thereof.

Optionally, a surfactant may be introduced to the saccharification reactor to enhance hydrolysis of the cellulose. In some embodiments, the surfactant comprises a lignosulfonate or other biomass-derived surfactant.

In some embodiments, the process further comprises removing ash present in the waste stream, prior to step (b). Removal of ash may be desirable when the waste stream contains high concentrations of ash, or to reduce ash in the recovered non-biomass component.

In some embodiments, the yield of the glucose in step (c) is at least 70% of theoretical, based on content of the cellulose in the waste stream. In preferred embodiments, the yield of the glucose in step (c) is at least 80% of theoretical, based on content of the cellulose in the waste stream.

In some embodiments, the yield of the non-biomass component in step (d) is at least 90% of theoretical, based on content of the non-biomass component in the waste stream. In certain embodiments, the process comprises recovering essentially all of the non-biomass component in step (d).

The process may be configured to recover substantially all of the non-biomass component, or at least the non-biomass component that does not degrade, if any, during fractionation or other processing. In certain embodiments, the non-biomass component is not substantially degraded during the fractionating, in which case substantially all of the initially present non-biomass component may be recovered for reuse (or combustion, gasification, etc.).

The process further may include fermenting the glucose to a fermentation product, such as (but not limited to) ethanol.

In some embodiments, the process further comprises recovering residual cellulose that is not saccharified in step (b). This residual cellulose may include nanocellulose or a nanocellulose precursor. The residual cellulose is refined to form nanocellulose, in certain embodiments.

In some embodiments, hemicellulose is contained in the waste stream, and the process further comprises hydrolyzing the hemicellulose to produce hemicellulosic monomers. These hemicellulosic monomers may be combined with the glucose, if desired. Also, when lignin is contained in the waste stream, the process may further comprise recovering at least some of the lignin.

Other variations of the invention provide a business method for generating sugars and recycling a non-biomass component from a waste stream, the method comprising:

(a) obtaining a waste stream comprising cellulose and a non-biomass component, wherein the waste stream is derived from or associated with production of a cellulose-containing product comprising the non-biomass component;

(b) introducing the waste stream to a saccharification reactor under effective conditions to saccharify at least some of the cellulose to produce glucose, wherein the non-biomass component is not substantially degraded in the saccharification reactor;

(c) recovering the glucose;

(d) recovering at least some of the non-biomass component as a recovered stream; and

(e) recycling the recovered stream to a site associated with production of the cellulose-containing product.

In some embodiments of this method, steps (a)-(e) are conducted at a single location. In some embodiments, step (e) includes transporting the recovered stream to a separate site. In certain embodiments, the waste stream is generated at a first location or plurality of locations, steps (b)-(d) are conducted at a second location or plurality of locations, and in step (e), the site associated with production of the cellulose-containing product is a third location or plurality of locations.

The non-biomass component may be selected from the group consisting of metals, metal oxides, salts, organic compounds, inorganic compounds, oligomers, polymers, and combinations thereof. In certain embodiments, without limitation, the polymer is selected from the group consisting of polyethylene, polypropylene, polylactide, polyethylene glycol, polyethylene terephthalate, polyacrylic acid, polyurethanes, synthetic rubber, phenol formaldehyde resin, neoprene, nylon, polyvinyl chloride, polystyrene, polyacrylonitrile, silicone, and combinations thereof.

Business systems may be configured to carry out the methods described. Apparatus may be configured to carry out the processes described. The invention also includes products produced by the disclosed processes and methods.

### BRIEF DESCRIPTION OF THE FIGURE

FIG. 1 is an exemplary process-flow diagram, according to some embodiments of the invention for waste streams containing polymers.

### DETAILED DESCRIPTION OF SOME EMBODIMENTS

This description will enable one skilled in the art to make and use the invention, and it describes several embodiments, adaptations, variations, alternatives, and uses of the invention. These and other embodiments, features, and advantages of the present invention will become more apparent to those skilled in the art when taken with reference to the following detailed description of the invention in conjunction with any accompanying drawings.

As used in this specification and the appended claims, the singular forms “a,” “an,” and “the” include plural referents unless the context clearly indicates otherwise. Unless defined otherwise, all technical and scientific terms used herein have the same meaning as is commonly understood by one of ordinary skill in the art to which this invention belongs. All composition numbers and ranges based on percentages are weight percentages, unless indicated otherwise. All ranges of numbers or conditions are meant to encompass any specific value contained within the range, rounded to any suitable decimal point.

Unless otherwise indicated, all numbers expressing parameters, reaction conditions, concentrations of components, and so forth used in the specification and claims are to be understood as being modified in all instances by the term “about.” Accordingly, unless indicated to the contrary, the numerical parameters set forth in the following specification and attached claims are approximations that may vary depending at least upon a specific analytical technique.

The term “comprising,” which is synonymous with “including,” “containing,” or “characterized by” is inclusive or open-ended and does not exclude additional, unrecited elements or method steps. “Comprising” is a term of art used in claim language which means that the named claim elements are essential, but other claim elements may be added and still form a construct within the scope of the claim.

As used herein, the phase “consisting of” excludes any element, step, or ingredient not specified in the claim. When the phrase “consists of” (or variations thereof) appears in a clause of the body of a claim, rather than immediately following the preamble, it limits only the element set forth in that clause; other elements are not excluded from the claim as a whole. As used herein, the phase “consisting essentially of” limits the scope of a claim to the specified elements or method steps, plus those that do not materially affect the basis and novel characteristic(s) of the claimed subject matter.

With respect to the terms “comprising,” “consisting of,” and “consisting essentially of,” where one of these three terms is used herein, the presently disclosed and claimed subject matter may include the use of either of the other two terms. Thus in some embodiments not otherwise explicitly recited, any instance of “comprising” may be replaced by “consisting of” or, alternatively, by “consisting essentially of.”

The present invention, in some variations, is premised on the recognition that the AVAP® process (which is commonly owned with the assignee of this application) may be modified for the economic production of sugars from waste streams. In some variations, the invention is premised on the realization that when a waste material is rich in cellulose and does not contain large quantities of lignin and/or hemicellulose, digestion with cooking chemicals may not be necessary and the waste stream may be enzymatically hydrolyzed to sugars directly or indirectly, with acids or enzymes, for example.

Certain exemplary embodiments of the invention will now be described. These embodiments are not intended to limit the scope of the invention as claimed. The order of steps may be varied, some steps may be omitted, and/or other steps may be added. Reference herein to first step, second step, etc. is for illustration purposes only.

In some variations, the invention provides a process for generating sugars from a waste stream, the process comprising:

(a) providing a waste stream comprising cellulose and a non-biomass component;

(b) introducing the waste stream to a saccharification reactor under effective conditions to saccharify at least some of the cellulose to produce glucose, wherein the non-biomass component is not substantially degraded in the saccharification reactor;

(c) recovering or further processing the glucose; and

(d) recovering at least some of the non-biomass component.

As used herein, “waste streams” may vary widely. Examples include municipal solid waste streams, industrial waste streams, consumer waste, recycled materials, waste paper, waste paper products, and other products derived from pulp, in each case including at least one non-biomass component. The cellulose or lignocellulosic biomass component may be a pulp material (such as cellulose from Kraft, sulfite, or other pulping) or may be a raw biomass feedstock (such as wood or agricultural residue). A “non-biomass component” is any material that is not directly derived from, or contained natively in, biomass. The non-biomass component may be organic or inorganic. Various moisture levels may be associated with the waste stream. The waste stream need not be, but may be, relatively dry.

In some embodiments, the waste stream is derived from clothing manufacturing, in which case the cellulose may be in the form of cotton or other cellulose fibers. Various non-biomass components may be present, including dyes, inks, or natural or synthetic materials.

In some embodiments, the non-biomass component is selected from the group consisting of metals, metal oxides, salts, organic compounds, inorganic compounds, oligomers, polymers, and combinations thereof. In specific embodiments, the non-biomass component includes a polymer, such as (but not limited to) a polymer selected from the group consisting of polyethylene, polypropylene, polylactide, polyethylene glycol, polyethylene terephthalate, polyacrylic acid, polyurethanes, synthetic rubber, phenol formaldehyde resin, neoprene, nylon, polyvinyl chloride, polystyrene, polyacrylonitrile, silicone, and combinations thereof.

In some embodiments, step (b) utilizes cellulase enzymes. In these or other embodiments, step (b) utilizes an acid or base catalyst. Step (b) may be conducted at a solids concentration of from about 1 wt % to about 25 wt % on a dry basis, such as from about 2 wt % to about 10 wt % on a dry basis, for example.

The process may further comprise a refining step, prior to or during step (b), to reduce average particle size of the waste stream. In some embodiments, the refining step includes mechanical refining, chemical refining, thermomechanical refining, chemithermomechanical refining, or a combination thereof.

Optionally, a surfactant may be introduced to the saccharification reactor to enhance hydrolysis of the cellulose. In some embodiments, the surfactant comprises a lignosulfonate or other biomass-derived surfactant.

In some embodiments, the process further comprises removing ash present in the waste stream, prior to step (b). Removal of ash may be desirable when the waste stream contains high concentrations of ash, or to reduce ash in the recovered non-biomass component.

In some embodiments, the yield of the glucose in step (c) is at least 70% of theoretical, based on content of the cellulose in the waste stream. In preferred embodiments, the yield of the glucose in step (c) is at least 80% of theoretical, based on content of the cellulose in the waste stream.

In some embodiments, the yield of the non-biomass component in step (d) is at least 90% of theoretical, based on content of the non-biomass component in the waste stream. In certain embodiments, the process comprises recovering essentially all of the non-biomass component in step (d).

The process may be configured to recover substantially all of the non-biomass component, or at least the non-biomass component that does not degrade, if any, during fractionation or other processing. In certain embodiments, the non-biomass component is not substantially degraded during the fractionating, in which case substantially all of the initially present non-biomass component may be recovered for reuse (or combustion, gasification, etc.).

The process further may include fermenting the glucose to a fermentation product, such as (but not limited to) ethanol.

In some embodiments, the process further comprises recovering residual cellulose that is not saccharified in step (b). This residual cellulose may include nanocellulose or a nanocellulose precursor. The residual cellulose is refined to form nanocellulose, in certain embodiments.

In some embodiments, hemicellulose is contained in the waste stream, and the process further comprises hydrolyzing the hemicellulose to produce hemicellulosic monomers. These hemicellulosic monomers may be combined with the glucose, if desired. Also, when lignin is contained in the waste stream, the process may further comprise recovering at least some of the lignin.

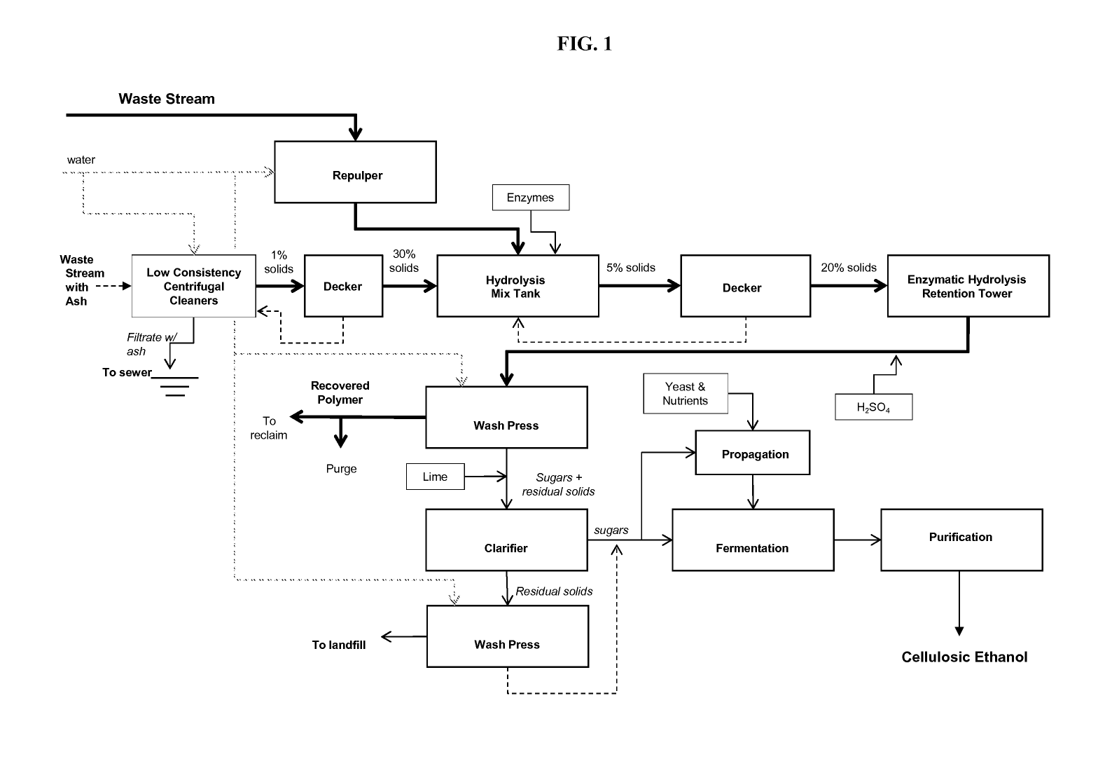

FIG. 1 is an exemplary process-flow diagram, according to some embodiments of the invention for waste streams containing polymers. In FIG. 1, a cellulose-containing waste stream is saccharified with enzymes to generate sugars (including glucose). A waste stream containing ash may optionally be processed, first with a centrifuge (for example) to remove ash. The sugars are fermented to ethanol, and the recovered polymer may be reclaimed for further reuse, or purged from the process.

Other variations of the invention provide a business method for generating sugars and recycling a non-biomass component from a waste stream, the method comprising:

(a) obtaining a waste stream comprising cellulose and a non-biomass component, wherein the waste stream is derived from or associated with production of a cellulose-containing product comprising the non-biomass component;

(b) introducing the waste stream to a saccharification reactor under effective conditions to saccharify at least some of the cellulose to produce glucose, wherein the non-biomass component is not substantially degraded in the saccharification reactor;

(c) recovering the glucose;

(d) recovering at least some of the non-biomass component as a recovered stream; and

(e) recycling the recovered stream to a site associated with production of the cellulose-containing product.

In some embodiments of this method, steps (a)-(e) are conducted at a single location. In some embodiments, step (e) includes transporting the recovered stream to a separate site. In certain embodiments, the waste stream is generated at a first location or plurality of locations, steps (b)-(d) are conducted at a second location or plurality of locations, and in step (e), the site associated with production of the cellulose-containing product is a third location or plurality of locations.

The non-biomass component may be selected from the group consisting of metals, metal oxides, salts, organic compounds, inorganic compounds, oligomers, polymers, and combinations thereof. In certain embodiments, without limitation, the polymer is selected from the group consisting of polyethylene, polypropylene, polylactide, polyethylene glycol, polyethylene terephthalate, polyacrylic acid, polyurethanes, synthetic rubber, phenol formaldehyde resin, neoprene, nylon, polyvinyl chloride, polystyrene, polyacrylonitrile, silicone, and combinations thereof.

In some embodiments, step (d) utilizes cellulase enzymes. In other embodiments, step (d) utilizes an acid or base catalyst. Catalyst or enzyme recycle may be employed.

In some embodiments, hemicellulose is contained in the lignocellulosic biomass, and the process further comprises hydrolyzing the hemicellulose to produce hemicellulosic monomers. These hemicellulosic monomers may be combined with the glucose, if desired. Also, when lignin is contained in the lignocellulosic biomass, the process may further comprise recovering at least some of the lignin. Generally speaking, the cellulose will have at least some hemicellulose and lignin present, although it is possible for the waste stream to include high-purity cellulose with essentially no detectible lignin and/or hemicellulose.

In preferred embodiments, the process further comprises recovering at least some of the non-biomass component. The process may be configured to recover substantially all of the non-biomass component, or at least the non-biomass component that does not degrade, if any, during fractionation or other processing. In certain embodiments, the non-biomass component is not substantially degraded during the fractionating, in which case substantially all of the initially present non-biomass component may be recovered for reuse (or combustion, gasification, etc.). Here, “degraded” means depolymerized, polymerized, or otherwise chemically reacted—that is, chemically degraded. There may be physical/mechanical changes to the non-biomass component, such as reduction of particle size, viscosity, etc.

Other variations of the present invention provide a process for generating sugars from a waste stream, the process comprising:

(a) providing a waste stream comprising cellulose and a non-biomass component;

(b) introducing the waste stream to a saccharification reactor under effective conditions to saccharify the cellulose to produce glucose, wherein the non-biomass component is not substantially degraded in the saccharification reactor;

(c) recovering at least some of the glucose; and

(d) recovering at least some of the non-biomass component.

Optionally, the waste stream is pretreated or digested prior to step (b). If such a pretreatment is performed, it preferably does not substantially degrade the non-biomass component. The pretreatment may be any known treatment including chemical or mechanical treatment. In some embodiments, the pretreatment is digestion in the presence of water, an acid catalyst (such as SO₂), and a solvent (such as ethanol). The solvent preferably facilitates a higher mass transfer rate of the acid catalyst into the lignocellulosic biomass, compared to the mass transfer rate of catalyst into the lignocellulosic biomass with water alone. For example, ethanol facilitates better SO₂ mass transfer because ethanol (with dissolved SO₂) is able to penetrate into biomass pores more efficiently than water.

Reaction conditions and operation sequences may vary widely. Some embodiments employ conditions described in U.S. Pat. No. 8,030,039, issued Oct. 4, 2011; U.S. Pat. No. 8,038,842, issued Oct. 11, 2011; U.S. Pat. No. 8,268,125, issued Sep. 18, 2012; and U.S. patent application Ser. Nos. 13/004,431; 12/234,286; 13/585,710; 12/250,734; 12/397,284; 12/304,046; 13/500,916; 13/626,220; 12/854,869; 61/732,047; 61/735,738; 61/739,343; 61/747,010; 61/747,105; 61/747,376; 61/747,379; 61/747,382; 61/747,408; and/or 61/827,827, including the prosecution histories thereof. Each of these commonly owned patent applications is hereby incorporated by reference herein in its entirety. In some embodiments, the process is a variation of the AVAP® process technology (or a portion thereof) which is commonly owned with the assignee of this patent application.

In some embodiments, the acid catalyst is present in a liquid-phase concentration of about 1 wt % to about 50 wt %, such as about 6 wt % to about 30 wt %, or about 9 wt % to about 20 wt %. The catalyst may be selected from the group consisting of sulfur dioxide, sulfur trioxide, sulfurous acid, sulfuric acid, sulfonic acid, lignosulfonic acid, elemental sulfur, polysulfides, and combinations or derivatives thereof.

In some variations, the invention provides a process for generating sugars from a waste stream, the process comprising:

(a) providing a waste stream comprising lignocellulosic biomass and a non-biomass component;

(b) in a digestor, treating the waste stream in the presence of a solvent for lignin, an acid catalyst, and water, to produce a liquor containing insoluble solids, dissolved hemicellulose (if hemicellulose is contained in the lignocellulosic biomass), and dissolved lignin (if lignin is contained in the lignocellulosic biomass), wherein the insoluble solids include cellulose and the non-biomass component;

(c) substantially removing the insoluble solids from the liquor; and

(d) saccharifying at least some of the insoluble solids to produce glucose.

Other variations of the present invention provide a business method for generating sugars and recycling a non-biomass component from a waste stream, the method comprising:

(a) obtaining a waste stream comprising cellulose and a non-biomass component, wherein the waste stream is derived from or associated with production of a cellulose-containing product comprising the non-biomass component;

(b) introducing the waste stream to a saccharification reactor under effective conditions to saccharify the cellulose to produce glucose, wherein the non-biomass component is not substantially degraded in the saccharification reactor;

(c) recovering at least some of the glucose;

(d) recovering at least some of the non-biomass component as a recovered stream; and

(e) recycling the recovered stream to a site associated with production of the cellulose-containing product.

In some method embodiments, steps (a)-(e) are conducted at a single site. In some embodiments, step (e) includes transporting the recovered stream to a separate site. In certain business scenarios, the waste stream is generated at a first site or plurality of sites, steps (b)-(d) are conducted at a second site or plurality of sites, and in step (e), the site associated with production of the cellulose-containing product is a third site or plurality of sites.

The non-biomass component may be selected from the group consisting of metals, metal oxides, salts, organic compounds, inorganic compounds, oligomers, polymers, and combinations thereof. In certain embodiments, without limitation, the polymer is selected from the group consisting of polyethylene, polypropylene, polylactide, polyethylene glycol, polyethylene terephthalate, polyacrylic acid, polyurethanes, synthetic rubber, phenol formaldehyde resin, neoprene, nylon, polyvinyl chloride, polystyrene, polyacrylonitrile, silicone, and combinations thereof.

Business systems may be configured to carry out the methods described. Apparatus may be configured to carry out the processes described. The invention also includes products produced by the disclosed processes and methods.

The sugars produced and recovered may be fermented to various products. The fermentation product may include an oxygenated compound, such as (but not limited to) oxygenated compounds selected from the group consisting of ethanol, propanol, butanol, pentanol, hexanol, heptanol, octanol, glycerol, sorbitol, propanediol, butanediol, butanetriol, pentanediol, hexanediol, acetone, acetoin, butyrolactone, 3-hydroxybutyrolactone, and any isomers, derivatives, or combinations thereof.

In some embodiments, the oxygenated compound is a C₃ or higher alcohol or diol, such as 1-butanol, isobutanol, 1,4-butanediol, 2,3-butanediol, or mixtures thereof.

The fermentation product may include a hydrocarbon, such as isoprene, farnasene, and related compounds.

Multiple fermentation products may be produced in a single fermentor, in co-product production or as a result of byproducts due to contaminant microorganisms. For example, during fermentation to produce lactic acid, ethanol is a common byproduct due to contamination (and vice-versa).

Multiple fermentation products may be produced in separate fermentors. In some embodiments, a first fermentation product, such as an organic acid, is produced from glucose (hydrolyzed cellulose) while a second fermentation product, such as ethanol, is produced from hemicellulose sugars. Or, in some embodiments, different fermentations are directed to portions of feedstock having varying particle size, crystallinity, or other properties.

In some embodiments, the fermentation product includes an enzymatically isomerized variant of at least a portion of the fermentable sugars. For example, the enzymatically isomerized variant may include fructose which is isomerized from glucose. In some embodiments, glucose, which is normally D-glucose, is isomerized with enzymes to produce L-glucose.

In some embodiments, the fermentation product includes one or more proteins, amino acids, enzymes, or microorganisms. Such fermentation products may be recovered and used within the process; for example, cellulase or hemicellulase enzymes may be used for hydrolyzing cellulose-rich solids or hemicellulose oligomers.

Some variations are premised on the recognition that the clean cellulose produced in these processes may be not only hydrolyzed to glucose, but also recovered as a cellulose pulp product, intermediate, or precursor (such as for nanocellulose).

In some embodiments, the cellulose-rich material is further processed into one more cellulose products. Cellulose products include market pulp, dissolving pulp (also known as α-cellulose), fluff pulp, purified cellulose, paper, paper products, and so on. Further processing may include bleaching, if desired. Further processing may include modification of fiber length or particle size, such as when producing nanocellulose or nanofibrillated or microfibrillated cellulose. It is believed that the cellulose produced by this process is highly amenable to derivatization chemistry for cellulose derivatives and cellulose-based materials such as polymers.

When hemicellulose is present in the waste stream, all or a portion of the liquid phase contains hemicellulose sugars and soluble oligomers. It is preferred to remove most of the lignin (if present) from the liquid, as described above, to produce a fermentation broth which will contain water, possibly some of the solvent for lignin, hemicellulose sugars, and various minor components from the digestion process. This fermentation broth can be used directly, combined with one or more other fermentation streams, or further treated. Further treatment can include sugar concentration by evaporation; addition of glucose or other sugars (optionally as obtained from cellulose saccharification); addition of various nutrients such as salts, vitamins, or trace elements; pH adjustment; and removal of fermentation inhibitors such as acetic acid and phenolic compounds. The choice of conditioning steps should be specific to the target product(s) and microorganism(s) employed.

In some embodiments, hemicellulose sugars are not fermented but rather are recovered and purified, stored, sold, or converted to a specialty product. Xylose, for example, can be converted into xylitol.

When lignin is present in the waste stream, a lignin product can be readily obtained from a liquid phase using one or more of several methods. One simple technique is to evaporate off all liquid, resulting in a solid lignin-rich residue. This technique would be especially advantageous if the solvent for lignin is water-immiscible. Another method is to cause the lignin to precipitate out of solution. Some of the ways to precipitate the lignin include (1) removing the solvent for lignin from the liquid phase, but not the water, such as by selectively evaporating the solvent from the liquid phase until the lignin is no longer soluble; (2) diluting the liquid phase with water until the lignin is no longer soluble; and (3) adjusting the temperature and/or pH of the liquid phase. Methods such as centrifugation can then be utilized to capture the lignin. Yet another technique for removing the lignin is continuous liquid-liquid extraction to selectively remove the lignin from the liquid phase, followed by removal of the extraction solvent to recover relatively pure lignin.

Lignin produced in accordance with the invention can be used as a fuel. As a solid fuel, lignin is similar in energy content to coal. Lignin can act as an oxygenated component in liquid fuels, to enhance octane while meeting standards as a renewable fuel. The lignin produced herein can also be used as polymeric material, and as a chemical precursor for producing lignin derivatives. The sulfonated lignin may be sold as a lignosulfonate product, or burned for fuel value.

The present invention also provides systems configured for carrying out the disclosed processes, and compositions produced therefrom. Any stream generated by the disclosed processes may be partially or completed recovered, purified or further treated, and/or marketed or sold.

In this detailed description, reference has been made to multiple embodiments of the invention and non-limiting examples relating to how the invention can be understood and practiced. Other embodiments that do not provide all of the features and advantages set forth herein may be utilized, without departing from the spirit and scope of the present invention. This invention incorporates routine experimentation and optimization of the methods and systems described herein. Such modifications and variations are considered to be within the scope of the invention defined by the claims.

All publications, patents, and patent applications cited in this specification are herein incorporated by reference in their entirety as if each publication, patent, or patent application were specifically and individually put forth herein.

Where methods and steps described above indicate certain events occurring in certain order, those of ordinary skill in the art will recognize that the ordering of certain steps may be modified and that such modifications are in accordance with the variations of the invention. Additionally, certain of the steps may be performed concurrently in a parallel process when possible, as well as performed sequentially.

Therefore, to the extent there are variations of the invention, which are within the spirit of the disclosure or equivalent to the inventions found in the appended claims, it is the intent that this patent will cover those variations as well. The present invention shall only be limited by what is claimed.

What is claimed is:

1. A process for generating sugars from a waste stream, said process comprising:
(a) providing a waste stream comprising cellulose and a non-biomass component;
(b) introducing said waste stream to a saccharification reactor under effective conditions to saccharify at least some of said cellulose to produce glucose, wherein said non-biomass component is not substantially degraded in said saccharification reactor;
(c) recovering or further processing said glucose; and
(d) recovering at least some of said non-biomass component.

2. The process of claim 1, wherein said non-biomass component is selected from the group consisting of metals, metal oxides, salts, organic compounds, inorganic compounds, oligomers, polymers, and combinations thereof.

3. The process of claim 2, wherein said non-biomass component includes a polymer.

4. The process of claim 3, wherein said polymer is selected from the group consisting of polyethylene, polypropylene, polylactide, polyethylene glycol, polyethylene terephthalate, polyacrylic acid, polyurethanes, synthetic rubber, phenol formaldehyde resin, neoprene, nylon, polyvinyl chloride, polystyrene, polyacrylonitrile, silicone, and combinations thereof.

5. The process of claim 1, wherein said waste stream is derived from clothing manufacturing.

6. The process of claim 1, wherein step (b) utilizes cellulase enzymes.

7. The process of claim 1, wherein step (b) utilizes an acid or base catalyst.

8. The process of claim 1, wherein step (b) is conducted at a solids concentration of from about 1 wt % to about 25 wt % on a dry basis.

9. The process of claim 8, wherein step (b) is conducted at a solids concentration of from about 2 wt % to about 10 wt % on a dry basis.

10. The process of claim 1, said process further comprising a refining step, prior to or during step (b), to reduce average particle size of said waste stream.

11. The process of claim 10, wherein said refining step includes mechanical refining, chemical refining, thermomechanical refining, chemithermomechanical refining, or a combination thereof.

12. The process of claim 1, wherein a surfactant is introduced to said saccharification reactor to enhance hydrolysis of said cellulose.

13. The process of claim 12, wherein said surfactant comprises a lignosulfonate.

14. The process of claim 1, said process further comprising removing ash present in said waste stream, prior to step (b).

15. The process of claim 1, wherein yield of said glucose in step (c) is at least 70% of theoretical, based on content of said cellulose in said waste stream.

16. The process of claim 15, wherein said yield of said glucose in step (c) is at least 80% of theoretical, based on content of said cellulose in said waste stream.

17. The process of claim 1, wherein yield of said non-biomass component in step (d) is at least 90% of theoretical, based on content of said non-biomass component in said waste stream.

18. The process of claim 17, said process comprising recovering essentially all of said non-biomass component in step (d).

19. The process of claim 1, said process further comprising fermenting said glucose to a fermentation product.

20. The process of claim 1, said process further comprising recovering residual cellulose that is not saccharified in step (b).

21. The process of claim 20, wherein said residual cellulose comprises nanocellulose.

22. The process of claim 20, wherein said residual cellulose is refined to form nanocellulose.

23. A business method for generating sugars and recycling a non-biomass component from a waste stream, said method comprising:
(a) obtaining a waste stream comprising cellulose and a non-biomass component, wherein said waste stream is derived from or associated with production of a cellulose-containing product comprising said non-biomass component;
(b) introducing said waste stream to a saccharification reactor under effective conditions to saccharify at least some of said cellulose to produce glucose, wherein said non-biomass component is not substantially degraded in said saccharification reactor;
(c) recovering said glucose;
(d) recovering at least some of said non-biomass component as a recovered stream; and
(e) recycling said recovered stream to a site associated with production of said cellulose-containing product.

24. The method of claim 23, wherein steps (a)-(e) are conducted at a single location.

25. The method of claim 23, wherein said waste stream is generated at a first location or plurality of locations, steps (b)-(d) are conducted at a second location or plurality of locations, and in step (e), said site associated with production of said cellulose-containing product is a third location or plurality of locations.
